(19) United States
(12) Patent Application Publication
(10) Pub. No.: US 2007/0286814 A1
(43) Pub. Date: Dec. 13, 2007

(54) STABLE AEROSOL PHARMACEUTICAL FORMULATIONS

(75) Inventors: Rajesh Sawant, Navi Mumbai (IN); Sunil Parab, Goa (IN)

Correspondence Address: MICHAEL BEST & FRIEDRICH LLP, ONE SOUTH PINCKNEY STREET, P O BOX 1806, MADISON, WI, 53701, US

(73) Assignee: Medispray Laboratories Pvt. Ltd., Goa (IN)

(21) Appl. No.: 11/533,857

(22) Filed: Sep. 21, 2006

(30) Foreign Application Priority Data
Jun. 12, 2006 (IN) 918/MUM/2006

(51) Int. Cl. A61K 31/138; A61K 9/12

(52) U.S. Cl. 424/45; 514/651

(57) ABSTRACT

The present invention provides a stable aerosol pharmaceutical formulation of a beta-agonist, an anticholinergic, or a combination thereof in combination with a cosolvent and optionally a surfactant. The invention also provides a method of making the stable aerosol pharmaceutical formulation and methods of treating bronchoconstriction, asthma and related conditions with the stable aerosol pharmaceutical formulation of the present invention.

### CROSS-REFERENCE TO RELATED APPLICATIONS

This application claims foreign priority benefits to Indian Application No. 918/MUM/2006, filed Jun. 12, 2006, and incorporated herein by reference in its entirety.

### BACKGROUND

Asthma is chronic inflammatory disease affecting about 20 million to 35 million persons worldwide, in which the patient suffers episodes of reversible airway obstruction. Asthma is generally treated by administration of anti-inflammatory drugs or bronchodilators. Anti-inflammatory drugs useful for the treatment of asthma include corticosteroids, mast cell stabilizers, and leukotriene inhibitors. Bronchodilators include beta-agonists, anticholinergics, and methylxanthines. Beta-agonists can be used to treat exercise-induced asthma. Beta-agonists can be combined with other classes of drugs like corticosteroids, anti-cholinergics and leukotriene inhibitors. The combination of a beta-agonist, such as albuterol, and an anticholinergic, such as ipratropium, has proven to be highly effective because the drugs provide bronchodilation by different mechanism of action.

Many asthma drugs are administered through inhalation. Suitable inhalation devices include metered dose inhalers (“MDIs”), dry powder inhalers and nebulizers. Metered dose inhalers conventionally contain one or more liquefied chlorofluorocarbons (“CFCs”) as propellant. Such materials are suitable for use in such applications since they have the right vapor pressures (or can be mixed in the right proportions to achieve a vapor pressure in the right range) and are essentially taste and odor-free.

One such CFC-containing metered dose inhaler is Combivent® Inhalation Aerosol which contains a microcrystalline suspension of ipratropium bromide and albuterol sulfate in a pressurized metered-dose aerosol unit for oral inhalation administration.

Due to environmental concerns, it is now desirable to use hydrofluoroalkane (“HFA”) propellants instead of CFCs in metered dose inhalers containing asthma drugs. For example, US20040184994 relates to a formulation comprising water in an amount of about 0.13 to about 0.18 percent (w/w) of the product formulation, one or more active ingredients and one or more excipients, wherein the preferred active ingredients are ipratropium and albuterol and the excipients are citric acid, ethanol and polyvinylpyrrolidone (“PVP”).

US20050085445 relates to a metered-dose aerosol inhaler composition, which contains a) at least one pharmaceutical active ingredient, b) at least one propellant, c) at least one native or modified cyclodextrin, d) at least one hydrophilic additive, and e) optionally ethanol.

US20050089478 relates to a formulation comprising formoterol and budesonide for use in the treatment of respiratory diseases. The composition further contains HFA-227 propellant, PVP and polyethylene glycol (“PEG”), preferably PVP K25 and PEG 1000.

However, MDI formulations containing HFA propellants do not have suspension characteristics as good as those formulations containing CFC Propellants. For example, an MDI formulation containing the beta-agonist albuterol sulfate and an anticholinergic agent, such as ipratropium bromide or tiotropium, with an HFA propellant is not a stable suspension and either quickly sediments or forms an emulsion.

For this reason, it is difficult to obtain a stable suspension using an HFA propellant. As a result, cosolvents such as water and alcohols (ethanol) have been used in combination with beta-agonist agents and anticholinergic agents in formulations containing HFA propellants. Unfortunately, the use of these cosolvents also leads to stability problems and other issues, such as agglomeration/increased particle size of the active agents, which are also undesirable.

### SUMMARY

The present invention provides a stable metered dose inhaler (“MDI”) formulation containing a pharmaceutically active agent with an HFA propellant along with suitable excipients. Surprisingly, it was found that a stable MDI formulation is formed when cosolvents other than alcohol, like polyethylene glycol (“PEG”), propylene glycol, isopropyl myristrate or glycerol are used, optionally in combination with a surfactant. The present invention thus provides a stable metered dose inhalation formulation comprising a beta-agonist and an anticholinergic with an HFA propellant and other suitable excipients. The formulation of the present invention does not form agglomerates. The present invention also provides a process for manufacture of a metered dose inhalation formulation.

The present invention is also a method for the treatment of bronchoconstriction, bronchospasm, asthma and related disorders thereof, which method comprises administering to a patient in need thereof an effective amount of a metered dose inhalation formulation according to the present invention.

Other aspects of the invention will become apparent by consideration of the detailed description and examples.

### DETAILED DESCRIPTION

The present invention provides a stable aerosol pharmaceutical formulation. More specifically, the stable aerosol pharmaceutical formulation contains a pharmaceutically active agent in combination with a hydrofluoroalkane (“HFA”) propellant and other suitable excipients.

As discussed earlier, aerosol formulations traditionally contained CFC propellants. Due to environmental concerns, HFA propellants are now preferred over CFC propellants. As will be understood by those skilled in the art, suitable HFA propellants for use in the present invention include, but are not limited to, 1,1,1,2-tetrafluoroethane (HFA-134a) and 1,1,1,2,3,3,3-heptafluoropropane (HFA-227).

The formulations of the present invention are suitable for use in MDIs. MDIs are compact drug delivery systems that use a liquefied propellant to atomize a precisely metered volume of a pharmaceutical formulation into particles, which are small enough to penetrate deep into the patient's lungs. MDIs allow for targeted delivery of drug to the desired site of the therapeutic effect—the lung.

The formulation of the present invention also includes a cosolvent, such as polyethylene glycol (“PEG”), propylene glycol, isopropyl myristrate or glycerol. Suitably, the cosolvent is PEG in liquid form, such as PEG 200 to PEG 400. The cosolvent can be present in a range of about 0.05 to about 15% by weight of the formulation. Suitably, the cosolvent is present in a range of about 0.05 to about 1% or about 0.05% to about 0.3%.

Further, it was found that when an optional surfactant is used along with the cosolvent, the surfactant maintains the homogeneity of the suspension and also acts as a lubricant for the smooth functioning of the valve on the MDI. Suitable surfactants include, but are not limited to, polyvinylpyrrolidone (“PVP”), sorbitan trioleate, oleic acid, citric acid, and polyoxyethylene(4)lauryl ether (Brij 30®). Suitably the surfactant is PVP, such as PVP K25 or PVP K30. The surfactant can be present in a range of about 0.00001% to about 10% by weight of the formulation. Suitably, the surfactant is present in a range of about 0.00001% to about 0.1% or about 0.0001% to about 0.001%.

Suitably, the formulations of the present invention are substantially free of water and alcohol. The term “substantially free of” means that the formulation contains less than about 5% water or alcohol by weight of the formulation. More suitably, the formulations contain less than about 3%, less than about 1%, less than about 0.5%, less than 0.1% or less than about 0.05% water or alcohol. Suitably, the formulations of the present invention contain no water or alcohol.

Surprisingly, it was also discovered that the use of polyoxyethylene sorbitan monolaurate without additional cosolvent provides a stable formulation. Thus, the present invention further provides a pharmaceutical formulation comprising an anticholinergic agent, a beta-agonist agent, polyoxyethylene sorbitan monolaurate, and a hydrofluoroalkane propellant. The polyoxyethylene sorbitan monolaurate can be present in a range of about 0.00001% to about 10% by weight of the formulation. Suitably, the polyoxyethylene sorbitan monolaurate is present in a range of about 0.00001% to about 0.1% or about 0.0001% to about 0.001%. More suitably, the polyoxyethylene sorbitan monolaurate is present in an amount of about 0.05% by weight of the formulation.

Pharmaceutically active agents useful in the present invention include one or more of drugs selected from the class of beta-agonists agents and anticholinergic agents. The terms “beta-agonist agent” or “beta-agonist” or “anticholinergic agent” are used in broad sense to include not only the beta-agonist or anticholinergic agent per se but also their pharmaceutically acceptable salts, pharmaceutically acceptable solvates, pharmaceutically acceptable hydrates, pharmaceutically acceptable enantiomers, pharmaceutically acceptable derivatives, pharmaceutically acceptable enantiomers, pharmaceutically acceptable polymorphs, pharmaceutically acceptable prodrugs, etc.

Beta-agonist agents useful in the present invention include, but are not limited to, albuterol, formoterol, levalbuterol, pirbuterol and salmeterol. The international name for albuterol is salbutamol. Suitable pharmaceutically acceptable salts of the beta-agonists include, but are not limited to the hydrochloride, sulfate, maleate, tartrate, and citrate salts. Suitably, the beta-agonist is albuterol or albuterol sulfate.

Anticholinergic agents useful in the present invention include, but are not limited to, ipratropium and tiotropium. Suitable pharmaceutically acceptable salts of the anticholinergic agents include, but are not limited to, the halide salts such as bromide, chloride and iodide. Suitably, the anticholinergic agent is ipratropium or ipratropium bromide or ipratropium bromide monohydrate.

The present invention also provides a method to manufacture a stable aerosol formulation according to the present invention. If used, the surfactant is dissolved in the cosolvent. The resulting solution is then mixed with an HFA propellant. If the surfactant is not used, the cosolvent is mixed with an HFA propellant. The pharmaceutically active agent is homogenized with additional HFA propellant to form a homogenized suspension. The homogenized suspension of active ingredients and solution of cosolvent and HFA propellant are mixed to form a second homogeneous suspension. The homogeneous second suspension is then placed in a precrimped canister or other container suitable for use as a metered dose inhaler.

For example, surfactant PVP K25 is dissolved in cosolvent PEG200/400 to make a clear solution. A quantity of HFA-227 propellant is added to the clear solution. A homogenized suspension of the ipratropium bromide and albuterol sulfate and additional HFA-227 propellant is formed. The homogenized suspension is added to the solution of PVP K25, PEG200/400 and HFA-227 to form a second homogeneous suspension. The resulting second suspension is then placed in a precrimped canister or other container suitable for use as a metered dose inhaler

The present invention further provides a method for the treatment of bronchoconstriction, bronchospasm, asthma and related disorders thereof, comprising administering to a patient in need thereof a stable aerosol formulation according to the present invention. Related disorders include, but are not limited to, chronic obstructive pulmonary disease, chronic bronchitis and emphysema.

The formulation of the present invention may be administered one, two, three or four times per day with one or more activations, e.g. two, three or four activations, of the metering valve per administration to treat bronchoconstriction, asthma and related disorders thereof. Up to about twelve inhalations of the pharmaceutical formulation of the present invention may be administered per 24 hour period.

Suitably, each actuation of the metering valve delivers about 21 μg of an anticholinergic agent, such as ipratropium bromide monohydrate and about 120 μg of a beta-agonist agent, such as albuterol sulfate from the metered dose inhaler. Suitably, each container or metered dose inhaler canister contains about 200 inhalations. As one skilled in the art is aware, the dose may be adjusted depending on the therapeutic objective of the use of the active agents and the age and condition of the patient.

We observed that some aerosol drugs tend to adhere to the inner surfaces, i.e., walls of the cans and valves, of the MDI. This can lead to the patient getting significantly less than the prescribed amount of the active agent upon each activation of the MDI. Coating the inner surface of the container with a suitable polymer can reduce this adhesion problem. Suitable coatings include, but are not limited to, fluorocarbon copolymers such as FEP-PES (fluorinated ethylene propylene and polyethersulphone) and PFA-PES (perfluoroalkoxyalkane and polyethersulphone), epoxy and ethylene.

Also, during storage, moisture can enter the MDI mainly through the crimped area of the valve and through the stem by diffusion. To reduce the amount of moisture entering the MDI, the metering valve is suitably comprised of a butyl elastomer.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

The following examples are for the purpose of illustration of the invention only and are not intended in any way to limit the scope of the present invention.

### EXAMPLE 1

The surfactant was dissolved in the cosolvent by sonication/homogenization for 10 minutes. This solution was mixed for 10-25 minutes in a mixing vessel with approximately 10%-15% of the total amount of propellant required for the batch. If the surfactant was not used, only the cosolvent was mixed with the propellant.

The active agents were homogenized in a separate homogenizer with approximately 5-15% of the total amount of propellant required for the batch. The homogenization speed and time ranged from 1000-1500 RPM for 15 to 30 minutes. The resulting homogenized suspension was transferred from the homogenizer to the mixing vessel through a double diaphragm pump. The remaining quantity of the propellant was then added to the mixing vessel.

The suspension was then mixed under constant stirring of 200-300 RPM for not less than 20 minutes and kept under recirculation through the double diaphragm pump.

Formulation 1

| | Qty/can |
|---|---|
| Ipratropium Bromide monohydrate | 5.04 mg |
| Albuterol Sulfate | 28.8 mg |
| Sorbitan Trioleate 0.00002% | 0.00408 mg |
| PEG 200 0.05% | 10.2 mg |
| HFA-227 | 20.4 gm |

Formulation 2

| | Qty/can |
|---|---|
| Ipratropium Bromide monohydrate | 5.04 mg |
| Albuteol Sulfate | 28.8 mg |
| Sorbitan Trioleate 0.00004% | 0.00816 mg |
| PEG 200 0.05% | 10.2 mg |
| HFA-227 | 20.4 gm |

Formulation 3

| | Qty/can |
|---|---|
| Ipratropium Bromide monohydrate | 5.04 mg |
| Albuterol Sulfate | 28.8 mg |
| Sorbitan Trioleate 0.05% | 10.2 mg |
| PEG 200 0.05% | 10.2 mg |
| HFA-227 | 20.4 gm |

Formulation 4

| | Qty/can |
|---|---|
| Ipratropium Bromide monohydrate | 5.04 mg |
| Albuterol Sulfate | 28.8 mg |
| PVP K 25 0.00001% | 0.00204 mg |
| PEG 200 0.1% | 20.4 mg |
| HFA-227 | 20.4 gm |

Formulation 5

| | Qty/can |
|---|---|
| Ipratropium Bromide monohydrate | 5.04 mg |
| Albuterol Sulfate | 28.8 mg |
| PVP K 25 0.00001% | 0.00204 mg |
| PEG 200 1% | 204 mg |
| HFA-227 | 20.2 gm |

Formulation 6

| | Qty/can |
|---|---|
| Ipratropium Bromide monohydrate | 5.04 mg |
| Albuterol Sulfate | 28.8 mg |
| PVP K 25 0.001% | 0.204 mg |
| PEG 200 0.3% | 61.2 mg |
| HFA-227 | 20.2 gm |

Formulation 7

| | Qty/can |
|---|---|
| Ipratropium Bromide monohydrate | 5.04 mg |
| Albuterol Sulfate | 28.8 mg |
| PVP K 30 0.1% | 20.4 mg |
| PEG 200 1% | 204 mg |
| HFA-227 | 20.2 gm |

Formulation 8

| | Qty/can |
|---|---|
| Ipratropium Bromide monohydrate | 5.04 mg |
| Albuterol Sulfate | 28.8 mg |
| PVP K 30 0.001% | 0.204 mg |
| PEG 200 0.3% | 61.2 mg |
| HFA-227 | 20.3 gm |

Formulation 9

| | Qty/can |
|---|---|
| Ipratropium Bromide monohydrate | 5.04 mg |
| Albuterol Sulfate | 28.8 mg |
| Oleic acid 0.001% | 0.204 mg |
| PEG200 1% | 204 mg |
| HFA-227 | 20.2 gm |

### EXAMPLE 2

The polyoxyethylene sorbitan monolaurate (Tween 20) was dissolved in the HFA propellent to form a solution. The active ingredients were homogenized with additional HFA propellant. The solution was then mixed with the homogenized suspension of active ingredients and HFA to form a second homogeneous suspension. The second suspension was then placed in a precrimped canister or other container suitable for use as an MDI. The formulation showed good stability.

Formulation 10

| | Qty/can |
|---|---|
| Ipratropium Bromide monohydrate | 5.04 mg |
| Albuterol Sulfate | 28.8 mg |
| Tween 20 (0.05%) | 10.2 mg |
| HFA-227 | 20.4 gm |

### EXAMPLE 3

Formulations made according to the process described above in Example 1 were tested for stability. Stability was determined by analyzing the particle size of the active ingredients using microscopy. An increase in particle size in examples 1 to 4 below indicated that the active ingredients were not as stable in the formulation. The results are shown below in Table 1.

TABLE 1
Effect of different combinations of cosolvent and surfactant on the suspension
characteristics and particle size of ipratropium bromide and albuterol sulfate.

| | Active | | | | Suspension | Particle size |
|---|---|---|---|---|---|---|
| | Ingredients | Cosolvent | Surfactant | Propellant | characteristics | observation |
| 1 | Ipratropium | — | — | HFA | Particles | Agglomeration |
| | (5.04 mg) | | | Propellant | remain in | |
| | Albuterol | | | (P134a or | homogeneous | |
| | sulfate | | | P227) | suspension for | |
| | (28.8 mg) | | | | less than 5 | |
| | | | | | seconds. | |
| 2 | Ipratropium | Alcohol | — | HFA | Particles | 70% particles |
| | (5.04 mg) | (1–5%) | | Propellant | remain in | between 10 to |
| | Albuterol | | | (P134a or | homogeneous | 12.5 microns |
| | sulfate | | | P227) | suspension for | |
| | (28.8 mg) | | | | about 10–15 | |
| | | | | | seconds. | |
| 3 | Ipratropium | Alcohol/ | — | HFA | Particles | 85% particles |
| | (5.04 mg) | Water | | Propellant | remain in | between 10 to |
| | Albuterol | (1–5%) | | (P134a or | homogeneous | 12.5 microns |
| | sulfate | | | P227) | suspension for | with |
| | (28.8 mg) | | | | about 10–15 | agglomeration |
| | | | | | seconds. | |
| 4 | Ipratropium | Alcohol | Polyvinyl- | HFA | Particles | 70% particles |
| | (5.04 mg) | (1–5%) | pyrrolidone | Propellant | remain in | between 10 to |
| | Albuterol | | or sorbitan | (P134a or | homogeneous | 12.5 microns |
| | sulfate | | trioleate or | P227) | suspension for | |
| | (28.8 mg) | | oleic acid or | | about 10–15 | |
| | | | citric acid or | | seconds. | |
| | | | polyoxy- | | | |
| | | | ethylene(4) | | | |
| | | | lauryl ether | | | |
| | | | (0.02–10%) | | | |
| 5 | Ipratropium | PEG200/ | — | HFA | Particles | 90% particles |
| | (5.04 mg) | 400 | | Propellant | remain in | below 2.5 |
| | Albuterol | (0.05–15%) | | (P134a or | homogeneous | microns & |
| | sulfate | | | P227) | suspension for | 10% between |
| | (28.8 mg) | | | | about 10–15 | 2.5 to 5 |
| | | | | | seconds. | microns |
| 6 | Ipratropium | PEG200/ | Polyvinyl- | HFA | Particles | 90% particles |
| | (5.04 mg) | 400 | pyrrolidone | Propellant | remain in | below 2.5 |
| | Albuterol | (0.05–15%) | or sorbitan | (P134a or | homogeneous | microns & |
| | sulfate | | trioleate or | P227) | suspension for | 10% between |
| | (28.8 mg) | | oleic acid or | | about 10–15 | 2.5 to 5 |
| | | | citric acid or | | seconds. | microns |
| | | | polyoxy- | | | |
| | | | ethylene(4) | | | |
| | | | lauryl ether | | | |
| | | | (0.00001–10%) | | | |
| 7 | Ipratropium | — | Polysorbate | HFA | Particles | 90% particles |
| | (5.04 mg) | | 20 or | Propellant | remain in | below 2.5 |
| | Albuterol | | Polysorbate | (P134a or | homogeneous | microns & |
| | sulfate | | 80 (0.01–0.05%) | P227) | suspension for | 10% between |
| | (28.8 mg) | | | | about 10–15 | 2.5 to 5 |
| | | | | | seconds. | microns |

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical formulation comprising an anticholinergic agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; a beta-agonist agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; a cosolvent selected from the group consisting of polyethylene glycol, propylene glycol, glycerol, and isopropyl myristrate; and a hydrofluoroalkane propellant.

2. The formulation of claim 1, wherein the formulation further comprises a surfactant.

3. The formulation of claim 2, wherein the surfactant is selected from the group consisting of polyvinylpyrrolidone, sorbitan trioleate, oleic acid, citric acid, and polyoxyethylene(4)lauryl ether.

4. The formulation of claim 1, wherein the anticholinergic is ipratropium or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof.

5. The formulation of claim 1, wherein the beta-agonist is albuterol or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof.

6. The formulation of claim 1, wherein the anticholinergic is ipratropium or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof and the beta-agonist is albuterol or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof.

7. The formulation of claim 1, wherein the cosolvent is present in an amount of about 0.05% to about 15% of the total weight of the formulation.

8. The formulation of claim 2, wherein the surfactant is present in an amount of about 0.00001% to about 10% of the total weight of the formulation.

9. The formulation of claim 1, wherein the formulation is substantially free of alcohol.

10. The formulation of claim 1, wherein the formulation is substantially free of water.

11. The formulation of claim 1, wherein the formulation contains less than about 0.1% water by weight of the formulation.

12. A pharmaceutical formulation comprising ipratropium bromide or its monohydrate, albuterol sulfate, about 0.05% to about 1% polyethylene glycol, about 0.00001% to about 0.1% polyvinylpyrrolidone, and a hydrofluoroalkane propellant.

13. A pharmaceutical formulation comprising an anticholinergic agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; a beta-agonist agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; polyoxyethylene sorbitan monolaurate; and a hydrofluoroalkane propellant.

14. The formulation of claim 13, wherein the polyoxyethylene sorbitan monolaurate is present in an amount of about 0.05% by weight of the formulation.

15. A method of making a pharmaceutical formulation comprising:
(a) dissolving a surfactant in a cosolvent selected from the group consisting of polyethylene glycol, propylene glycol, glycerol, and isopropyl myristrate to form a solution;
(b) mixing a hydrofluoroalkane propellant with the resulting solution;
(c) forming a homogenized suspension of an anticholinergic agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; a beta-agonist agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; and a hydrofluoroalkane propellant; and
(d) adding the homogenized suspension to the solution to form a second homogeneous suspension.

16. A method of making a pharmaceutical formulation comprising:
(a) dissolving polyoxyethylene sorbitan monolaurate in a hydrofluoroalkane propellant to form a solution;
(b) forming a homogenized suspension of an anticholinergic agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; a beta-agonist agent or pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs or pharmaceutically acceptable prodrugs thereof; and a hydrofluoroalkane propellant; and
(c) adding the homogenized suspension to the solution to form a second suspension.

17. A metered dose inhaler comprising the formulation of claim 1 and a canister coated with a polymer selected from the group consisting of a fluorocarbon polymer, an epoxy copolymer, and an ethylene copolymer.

18. The metered dose inhaler of claim 17, further comprising a sealing gasket comprising a butyl elastomer.

19. A metered dose inhaler comprising the formulation of claim 12 and a canister coated with a polymer selected from the group consisting of a fluorocarbon polymer, an epoxy copolymer, and an ethylene copolymer.

20. The metered dose inhaler of claim 19, further comprising a sealing gasket comprising a butyl elastomer.

21. A metered dose inhaler comprising the formulation of claim 13 and a canister coated with a polymer selected from the group consisting of a fluorocarbon polymer, an epoxy copolymer, and an ethylene copolymer.

22. The metered dose inhaler of claim 21, further comprising a sealing gasket comprising a butyl elastomer.

23. A method of treating bronchoconstriction, bronchospasm, asthma and related disorders comprising administering an effective amount of the formulation of claim 1 to patient in need thereof.

24. A method of treating bronchoconstriction, bronchospasm, asthma and related disorders comprising administering an effective amount of the formulation of claim 12 to patient in need thereof.

25. A method of treating bronchoconstriction, bronchospasm, asthma and related disorders comprising administering an effective amount of the formulation of claim 13 to patient in need thereof.
